(19) United States
(12) Patent Application Publication
(10) Pub. No.: US 2025/0275829 A1
(43) Pub. Date: Sep. 4, 2025

(54) ARTICULATION MECHANISMS FOR INSTRUMENTS, AND RELATED DEVICES AND METHODS

(71) Applicant: INTUITIVE SURGICAL OPERATIONS, INC., Sunnyvale, CA (US)

(72) Inventor: William A. Burbank, Sandy Hook, CT (US)

(73) Assignee: INTUITIVE SURGICAL OPERATIONS, INC., Sunnyvale, CA (US)

(21) Appl. No.: 19/064,110

(22) Filed: Feb. 26, 2025

Related U.S. Application Data
(60) Provisional application No. 63/559,296, filed on Feb. 29, 2024.

(51) Int. Cl. A61B 90/50

(52) U.S. Cl. CPC A61B 90/50; A61B 2090/506

(57) ABSTRACT

An articulation mechanism comprises a first link pivotably coupled at a first location radially offset in a first direction from a longitudinal axis, and to the distal portion at a second location radially offset in the first direction from the longitudinal axis. A first actuation element is configured to rotate the first link about a first axis at the first location and thereby cause rotation of the first link about a second axis at the second location. A second link is pivotably coupled at a third location radially offset in a second direction, opposite the first direction, from the longitudinal axis, and at a fourth location radially offset in the second direction from the longitudinal axis. A second actuation element configured to rotate the second link about a third axis extends through the third location and thereby causes rotation about a fourth axis extending through the fourth location.

### CROSS-REFERENCE TO RELATED APPLICATIONS

This application claims priority to U.S. Provisional Application No. 63/559,296, filed Feb. 29, 2024, which is incorporated herein by reference in its entirety.

### TECHNICAL FIELD

Aspects of the present disclosure relate to articulation mechanisms for use in instruments, and related devices and methods. For example, aspects of the present disclosure relate to articulation mechanisms to impart one or more degrees of freedom to instruments including, but not limited to, instruments used to perform surgical, diagnostic, therapeutic, and other medical or non-medical procedures. Further aspects of the disclosure relate to methods of configuring and operating such articulation mechanisms and instruments comprising such articulation mechanisms.

### INTRODUCTION

Various instruments, such as medical, including surgical, or other industrial instruments, often include shafts having articulatable portions that impart one or more degrees of freedom of movement to such instruments. Such articulatable portions can include one or more joints located along the shaft of the instrument. Each joint may articulate in one or more degrees of freedom (e.g., pitch and/or yaw), which may be the same or different as other joints.

One type of articulatable portion of an instrument can include two joints spaced from one another along a longitudinal axis of a shaft of the instrument and operably coupled to articulate in a coordinated fashion. The joints can be movable in a coordinated fashion in one or more degrees of freedom, such as pitch and/or yaw. Movement of both joints in the same degree of freedom but in opposite directions (relative to a longitudinal axis of the shaft and joints in a neutral, unarticulated state) results in a counter pivot mechanism to provide a generally parallel, offset arrangement between portions of the shaft proximal of and distal to the articulatable portion of the shaft between and including the two joints (e.g., in a manner similar to the relationship of upper arm, forearm, and hand when bending the elbow of an arm in one direction and the wrist of the arm in the opposite direction). Stated another way, movement of the joints in this manner can provide a generally coordinated translational movement of a portion of the instrument (e.g., distal to a distal-most joint of the articulatable portion) relative to another portion of the instrument (e.g., proximal to a proximal-most joint of the articulatable portion). This arrangement may be desirable, for example, to improve an angle of approach of an end effector of an instrument to a remote site, such as a remote site of a minimally invasive medical procedure.

As one example, for single-port type medical procedures in which multiple instruments are introduced through a single incision in a patient's body wall or through a natural orifice or lumen, articulation mechanisms providing such a counter pivot articulation mechanism between the different portions of the shaft can be used to space apart the multiple instruments to provide greater working room for each instrument (for example, to shift an instrument end effector for one or more of the multiple instruments from a common longitudinal axis that extends through the incision or orifice and along the instrument shaft during advancement through the incision or orifice). Optionally, one or more additional articulation mechanisms, such as a wrist joint coupling the end effector to the shaft of the instrument distal to the distal most joint of the counter pivot articulation mechanism, can then be articulated to further refine and direct the orientation of the end effector, including back toward a remote site. In this way, triangulation of the various instrument end effectors relative to the remote site may be achieved.

Articulation of such counter pivot articulation mechanisms can be actuated and controlled by one or more actuation elements (e.g., cables) coupled through various components to a manipulator system that receives inputs from a user, such as a surgeon or other operator, to position the instrument and end effector as desired. In some arrangements, these actuation elements may extend generally from a proximal portion of the instrument, distally through a distal-most joint of the articulation mechanism. Manipulator systems can include a teleoperated (e.g., computer-controlled) manipulator system to which an instrument is configured to be coupled and via which inputs are received from a user at a location remote from the manipulator system. Alternatively, a manipulator system can be configured for manual operation via various inputs at a handle or other mechanism attached to a proximal end portion of the instrument and designed to be operated by a user to control instrument motion.

There exists a need for mechanically robust articulation mechanisms and systems that impart articulation, such as counter pivoting articulation as described above and further below, to instruments. Additionally, there exists a need for such systems that possess desirable characteristics such as manufacturability, serviceability, and reliability in operation. Moreover, in applications in which space is limited, such as in minimally invasive medical (e.g., including surgical, diagnostic, and/or therapeutic) applications, there exists a need to provide systems that provide articulation to instruments that have sufficient strength and durability while also having sufficiently small dimensions.

### SUMMARY

Embodiments of the present disclosure may solve one or more of the above-mentioned problems and/or may demonstrate one or more of the above-mentioned desirable features. Other features and/or advantages may become apparent from the description that follows.

In accordance with at least one aspect of the present disclosure, a medical instrument includes a proximal portion, a distal portion, and an articulation mechanism coupling the proximal portion to the distal portion, a longitudinal axis extending along the proximal portion, distal portion, and an articulation mechanism. The articulation mechanism includes a first link comprising a proximal end portion pivotably coupled to the proximal portion at a first pivot axis and a distal end portion pivotably coupled to the distal portion, a first actuation element coupled to the proximal end portion of the first link and extending proximal to the first link along the longitudinal axis and providing actuation forces to the proximal end portion of the first link, a second link comprising a proximal end portion pivotably coupled to the proximal portion at a second pivot axis and a distal end portion pivotably coupled to the distal portion, and a second actuation element coupled to the proximal end portion of the second link and extending proximal to the second link along the longitudinal axis and providing actuation forces to the proximal end portion of the second link. The first link is pivotable relative to the proximal portion about the first pivot axis. The first actuation element is laterally offset from the first pivot axis such that actuation of the first actuation element creates a moment to rotate the first link about the first pivot axis and causes rotation of the first link about the proximal portion. The second link is pivotable relative to the proximal portion about the second pivot axis. The second actuation element is laterally offset from the second pivot axis such that actuation of the second actuation element creates a moment to rotate the second link about the second pivot axis and causes rotation of the second link about the proximal portion. Rotation of the first and second links about their respective axes occurs in a coordinated manner so as to translate the distal portion.

In another aspect of the present disclosure, a medical instrument includes a shaft extending along a longitudinal axis, the shaft comprising a proximal portion, a distal portion, and an articulation mechanism coupling the proximal portion and the distal portion. The medical instrument further comprises an actuation element terminating at and extending proximally from a proximal portion of the articulation mechanism. Actuation of the actuation element causes pivoting of the articulation mechanism in a first direction relative to the proximal portion and pivoting of the distal portion relative to the articulation mechanism in a second direction opposite the first direction.

In yet another aspect of the present disclosure, an articulation mechanism for coupling a proximal portion of an elongate instrument to a distal portion of an elongate instrument extending along a longitudinal axis comprises a first link comprising a proximal end portion and a distal end portion. The first link further comprises a first pivot coupling at the distal end portion configured to pivotably couple the first link to the distal portion at a first location radially offset in a first direction from the longitudinal axis, a second pivot coupling at the proximal end portion configured to pivotably couple the first link to the proximal portion at a second location radially offset in the first direction from the longitudinal axis, and a first actuation element coupled to the second pivot coupling and configured to transmit force to create a moment to rotate the first link about a first axis of rotation extending through the first location and about a second axis of rotation extending through the second location. The articulation mechanism further comprises a second link comprising a proximal end portion and a distal end portion. The second link further comprises a third pivot coupling at the distal end portion configured to pivotably couple to second link to the distal portion at a third location radially offset in a second direction, opposite the first direction, from the longitudinal axis, a fourth pivot coupling at the proximal end portion configured to pivotably couple the second link to the proximal portion at a fourth location radially offset in the second direction from the longitudinal axis, and a second actuation element coupled to the fourth pivot coupling and configured to transmit force to create a moment to rotate the second link about a third axis of rotation extending through the third location and about a fourth axis of rotation extending through the fourth location. The first, second, third, and fourth axes of rotation extend parallel to each other and perpendicular to the longitudinal axis. The distal member is translatable in two degrees of freedom in response to actuation of the first and second actuation elements.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are for example and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings,

FIG. 1 is a schematic, side view of an embodiment of an instrument comprising an articulation mechanism according to embodiments of the present disclosure.

FIG. 2 is a schematic, side view of an articulation mechanism illustrating principles of operation for articulation of an instrument including such an articulation mechanism according to some embodiments of the present disclosure.

FIG. 3 is a side view of an instrument comprising an articulation mechanism for articulation of the instrument according to an embodiment of the present disclosure.

FIG. 4 is a perspective, detailed view of the portion of the instrument labeled 4-4 in FIG. 3.

FIG. 5 is a cross-sectional view of the instrument of FIG. 3 along section 5-5.

FIG. 6 is another perspective, detailed view of the portion of the instrument labeled 6-6 in FIG. 3.

FIG. 7 is a cross-sectional view of the instrument of FIG. 3 along section 7-7.

FIG. 8 is a schematic, side view of another articulation mechanism to illustrate principles of operation of articulation of an instrument including such an articulation mechanism according to another embodiment of the present disclosure.

FIG. 9 is a perspective schematic view of a manipulator system according to some embodiments of the disclosure.

FIG. 10 is a partial schematic view of another embodiment of a manipulator system according to some embodiments of the present disclosure.

### DETAILED DESCRIPTION

Embodiments of the present disclosure relate to articulation mechanisms for instruments that are configured to provide compound articulation and translational movement, such as for example similar to that imparted by a parallel motion mechanism, though in some embodiments of the present disclosure the motion may be only approximately parallel between different segments of an instrument shaft and thus is more generally considered a counter pivot mechanism. In some embodiments, an articulation mechanism is positioned between a proximal portion of the instrument and a distal portion of the instrument, and actuation of the articulation mechanism results in lateral movement of the distal portion relative to a longitudinal axis of the instrument.

Various articulation mechanisms according to the present disclosure can be configured to produce movement of a distal portion relative to a proximal portion of an instrument with respect to the longitudinal axis in a single degree of freedom, such as pitch motion, or movement of the distal portion relative to the proximal portion in two degrees of freedom, such as pitch motion, yaw motion, and combined pitch and yaw motions. To facilitate motion in both pitch and yaw, articulation mechanisms according to the present disclosure can include link components that are coupled to the distal portion and the proximal portion such that links are movable in multiple degrees of freedom. For example, in some embodiments, an articulation mechanism includes four link assemblies that are coupled to each of the proximal portion and the distal portion in a manner that allows each link assembly to move in two degrees of freedom.

According to some arrangements, a distal portion of actuation elements (e.g., cables, actuation rods, or the like) are coupled to proximal end portions of links of an articulation mechanism, such that the actuation elements do not extend distally past their respective locations to which they couple to the articulation mechanism. In other words, control of the articulation mechanism is provided by actuation elements that extend only to a proximal end portion of the articulation mechanism and not to a distal end portion of the articulation mechanism. Such an articulation mechanism arrangement may have increased strength relative to articulation mechanisms that require actuation elements that extend to the distal end portion of the actuation mechanism (e.g., due to compliance of the actuation elements). The increased strength can allow the instrument to handle higher loads/forces, which may be useful for increased end effector loads, such as, for example, in certain stapler end effectors or higher gripping force instruments.

Such an arrangement may make the actuation elements less vulnerable to interference from structures or materials of the environment in which the articulation mechanism is used as compared to articulation mechanisms (such as pivoting joints) in which the actuation elements extend partially or entirely through the articulating portion from a location proximal to a location distal to or within the articulation portion.

Further, because the actuation elements in accordance with articulation mechanisms of the present disclosure operate within a non-articulating portion of the instrument, the actuation elements need not be flexible, and therefore more rigid actuation elements that can provide robust push and pull forces can be utilized. Mechanical advantage can be doubled with one side pulling while the opposite is pushing.

In addition, pivot coupling locations can be arranged near the perimeter of the instrument, which allows the moment arm distance from pivot to actuation element to be relatively large. This also can improve mechanical advantage.

In some arrangements, the articulation mechanisms are configured to impart the lateral movement of the distal portion in translation alone relative to a longitudinal axis of the instrument. In other embodiments, kinematic relationships between various components of the articulation mechanism are configured such that as the distal portion is moved laterally away from the longitudinal axis of the instrument, the distal portion also rotates (angles or articulates) toward the longitudinal axis (thus the orientation may not be perfectly parallel and so is considered a counter pivot between the two portions the articulation mechanism connects). This arrangement can reduce the total range of motion required to be provided by other articulation mechanisms of the instrument, such as a wrist joint located between the articulation mechanism and an end effector at a distal end portion of an instrument.

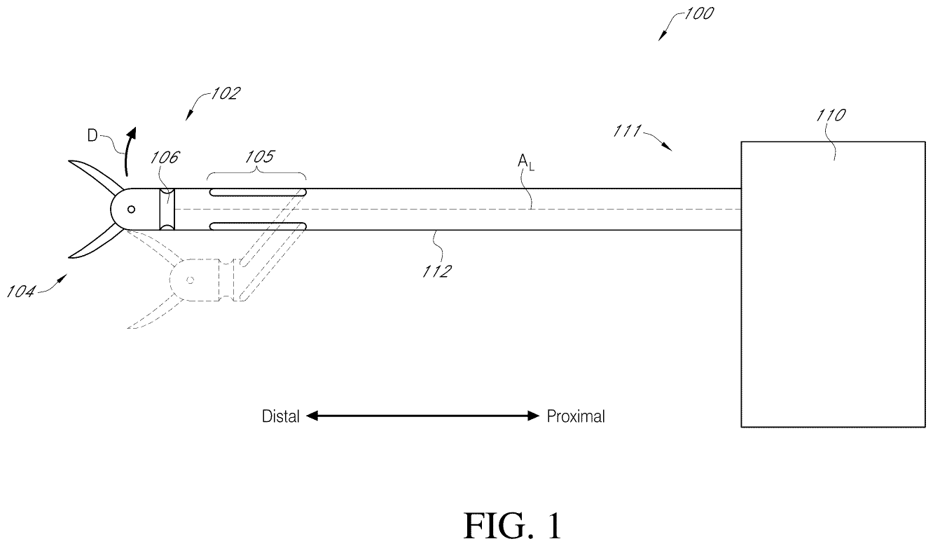

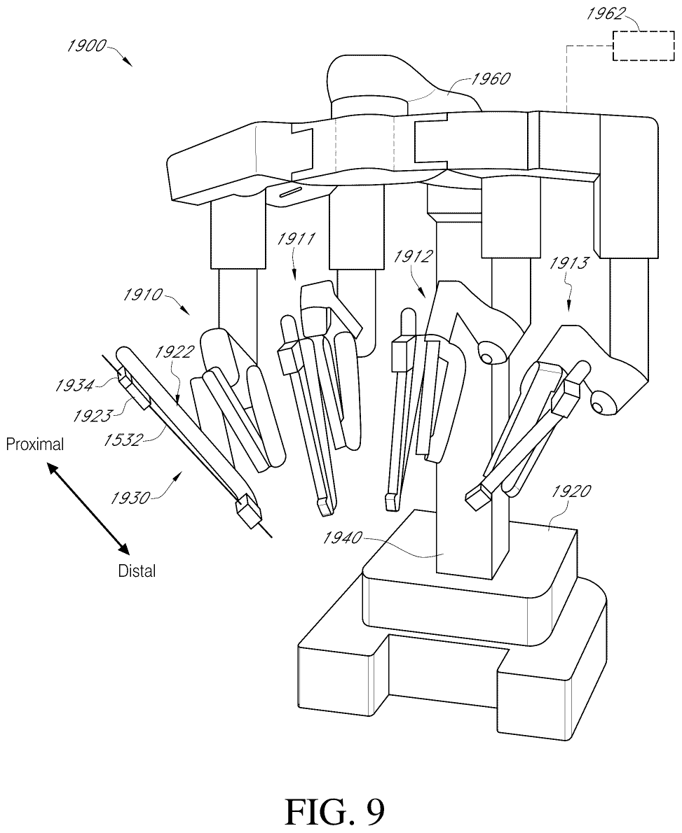

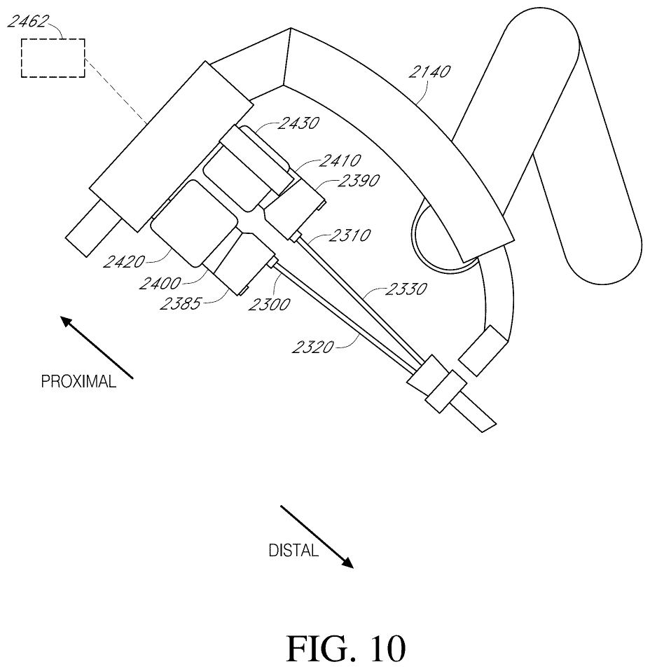

Referring now to FIG. 1, a schematic, side view of an elongate instrument 100 according to some embodiments of the disclosure is shown. The instrument 100 includes an end effector 104, a shaft 112 defining a longitudinal axis A_{L}, and a force transmission mechanism 110. The end effector 104 is located at a distal end portion 102 of the shaft 112. The end effector 104 can be configured to carry out a medical or non-medical (such as industrial) procedure. For example, the end effector 104 can include one or more tools such as gripping tools, staplers, shears, ligation clip appliers, electrosurgical tools, or other types of tools. The force transmission mechanism 110 is coupled to a proximal end portion 111 of the shaft 112. In some embodiments, the force transmission mechanism 110 may be coupled to a different portion of the shaft 112, for example, a middle portion of the shaft 112. The force transmission mechanism 110 can be operably coupled with a computer-controlled (e.g., teleoperated) surgical manipulator system, such as the manipulator systems described above and in further detail below in connection with FIGS. 9 and 10, and/or the force transmission mechanism 110 can be manually controlled with manually operated (e.g., handheld) actuators (not shown and as described above).

In the embodiment shown in FIG. 1, the instrument 100 includes an articulation mechanism 105 arranged along the shaft 112 between the end effector 104 and the transmission mechanism 110. Thus, the shaft 112 may be composed of multiple portions, with a relatively proximal portion and a relatively distal portion coupled by the articulation mechanism 105. Further, while the embodiment of FIG. 1 includes a single articulation mechanism 105, multiple articulation mechanisms can optionally be included along the length of the shaft 112 in any desired locations. As shown in FIG. 1, the articulation mechanism 105 can be positioned toward the distal end portion 102 of the shaft 112. However, the disclosure is not so limited and the articulation mechanism 105 can be positioned at any location along the shaft 112 without limitation.

As discussed further herein, the articulation mechanism 105 can be actuated to translate a portion of the instrument distal to the articulation mechanism] (e.g., the end effector 104, a wrist mechanism, or another portion of the instrument 100) laterally away from the longitudinal axis A_{L} of the instrument 100, such that the translated instrument portion is offset laterally from the longitudinal axis A_{L}. As also discussed further herein, the articulation mechanism 105 can be configured to provide multiple degrees of freedom. For example, the articulation mechanism 105 can be configured to offset the end effector 104 from the central longitudinal axis A_{L} in pitch, yaw, or a combination of pitch and yaw.

In various embodiments disclosed herein, another articulation mechanism can be provided to impart one or more degrees of freedom to the shaft 112 and/or end effector 104 may be provided in addition to the articulation mechanism 105. For example, in the embodiment of FIG. 1, the instrument 100 includes a wrist mechanism 106 located proximal of the end effector 104 and distal of the articulation mechanism 105. The wrist mechanism 106 can be configured to articulate in one or more degrees of freedom, such as one or both of pitch and yaw. The arrangement of FIG. 1 is intended to be illustrative, and embodiments having other types of articulation mechanisms to orient the end effector 104 relative to the instrument shaft 112, are considered within the scope of the disclosure.

The force transmission mechanism 110 may include one or more inputs (not shown) that are actuatable via a manual or computer-assisted (e.g., teleoperated) manipulator system, can actuate the end effector 104, such as to carry out a gripping, shearing, stapling, electrosurgery, or other procedure as those having ordinary skill in the art would be familiar with. Additionally, other drive inputs received at the transmission mechanism 110 can transmit force to actuate the actuation mechanism 105 and/or actuation mechanisms, such as wrist mechanism 106, to obtain a desired overall shape/orientation of the instrument from the transmission mechanism 110 to the end effector 104. For example, as discussed above, for procedures involving multiple instruments being inserted through a single incision in the body wall, the actuation mechanism 105 can be actuated to move the instrument portions distal of the actuation mechanism 105 (including the end effector 104) away from the longitudinal axis A_{L} while maintaining an orientation of the instrument portions distal of the actuation mechanism 105 generally parallel to the longitudinal axis AL, as shown in dashed lines in FIG. 1. If desired, the wrist mechanism 106 can be articulated to direct an orientation of the end effector 104 back towards the longitudinal axis A_{L} to access the worksite.

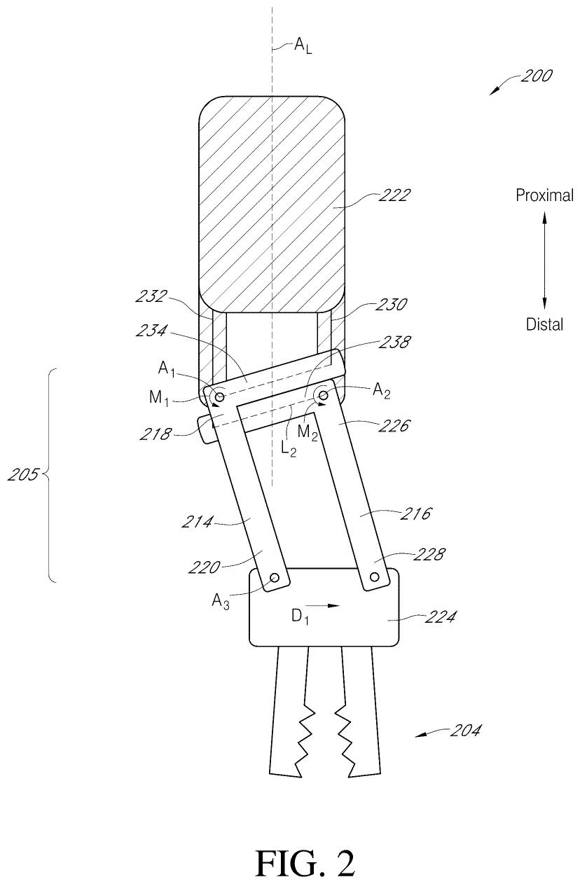

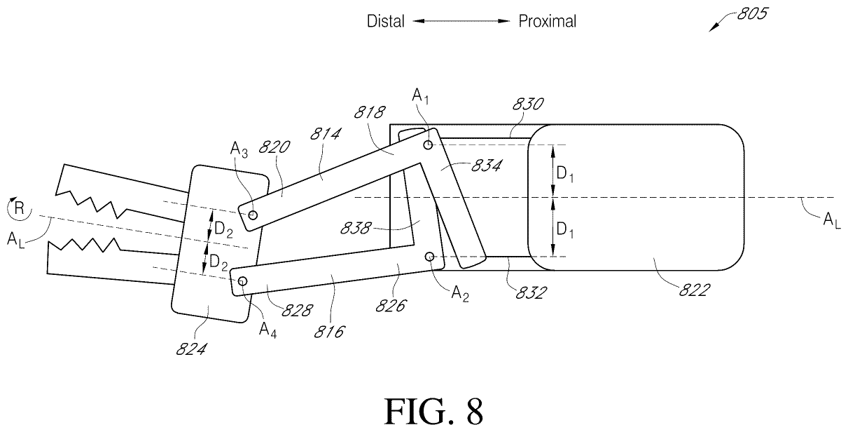

Turning now to FIG. 2, an articulation mechanism 205 that can be used as articulation mechanism 105 is depicted. The articulation mechanism 205 includes a first link 214 and a second link 216 extending generally along a longitudinal axis A_{L} of an instrument 200 to couple a proximal portion 222 of the instrument to a distal portion 224 (which is as an end effector 204 in FIG. 2 but could be a distal component to which an end effector is coupled, such as a wrist mechanism or other portion) of the instrument. The first link 214 has a proximal end portion 218 and a distal end portion 220. The first link 214 is rotatably coupled at the proximal end portion 218 to the proximal portion 222 (e.g., a proximal portion of a shaft such as the shaft 112 shown in FIG. 1) such that the first link 214 is rotatable about a first pivot axis A₁ on a first side of the longitudinal axis A_{L} (e.g., left side as depicted). The second link 216 has a proximal end portion 226 and a distal end portion 228. The proximal end portion 226 of the second link 216 is rotatably coupled to the proximal portion 222 so as to be rotatable about a second pivot axis A₂ opposite the first pivot axis A₁ relative to the longitudinal axis A_{L} (e.g., the second link 216 is on a right side of the longitudinal axis A_{L} as depicted). The distal end portions 220 and 228 of the first link 214 and the second link 216 are each rotatably coupled to the distal portion 224, such that the first link 214 and/or distal portion 224 are rotatable relative to each other about a third pivot axis A₃ and the second link 216 and/or distal portion 224 are rotatable relative to each other about a fourth pivot axis A₄ at the distal end portion 228 of the second link 216 to the distal portion 224. In a state of the pivot axes A₁, A₂, A₃, and A₄ in a neutral (e.g., non-rotated) position relative to the longitudinal axis A_{L}, the third pivot axis A₃ is on the same side of the longitudinal axis A_{L} as the first pivot axis A₁ (e.g., left side in FIG. 2), and the fourth pivot axis A₄ is on the same side of the longitudinal axis A_{L} as the second pivot axis A₂ (e.g., right side in FIG. 2). Further, the pivot axes A₁, A₂, A₃, and A₄ are equidistant from the longitudinal axis A_{L} in the neutral state of the articulation mechanism in which the proximal and distal portions 222, 224 are aligned along the longitudinal axis A_{L}, such that a first distance measured between the longitudinal axis A_{L} and the pivot axis A₁ is the same as a second distance measured between the longitudinal axis A_{L} and the pivot axis A₂. Other embodiments herein, such as the embodiment discussed in connection with FIG. 8, may include axes that have differing distances from the longitudinal axis to provide various kinematic advantages, as described further below. While the distal portion 224 as shown in FIG. 2 does not include a wrist mechanism (such as wrist mechanism 106 in FIG. 1 interposed between the articulation mechanism 205 and the end effector), the embodiment of FIG. 2 can include one or more additional articulation mechanisms, such as a wrist mechanisms or other joints between the articulation mechanism 205 and the end effector, in which case the articulation mechanism 205 would couple to another distal portion located proximally of the end effector.

In some implementations, actuation of the articulation mechanism 205 can be achieved by generating a moment to rotate the first link 214 and the second link 216 about their respective pivot axes A₁ and A₂ and thereby cause generally lateral movement of the distal portion 224 with respect to the longitudinal axis A_{L} while the proximal portion 222 remains in a generally neutral alignment with the longitudinal axis A_{L}. In addition, one or more actuation elements can be respectively coupled to the first link 214 and second link 216 in a manner such that application of tensile forces (e.g., pull-type actuation elements such as cables) and tensile and compressive forces (e.g., push-pull type actuation elements such as actuation rods) create moments to rotate the first link 214 and second link 216 about their respective pivot axes A₁ and A₂ at the proximal portion 222. For example, a first actuation element 230 can be coupled to the first link 214 at a location offset from the first pivot axis A₁, and a second actuation element 232 can be coupled to the second link 216 at a location offset from the second pivot axis A₂.

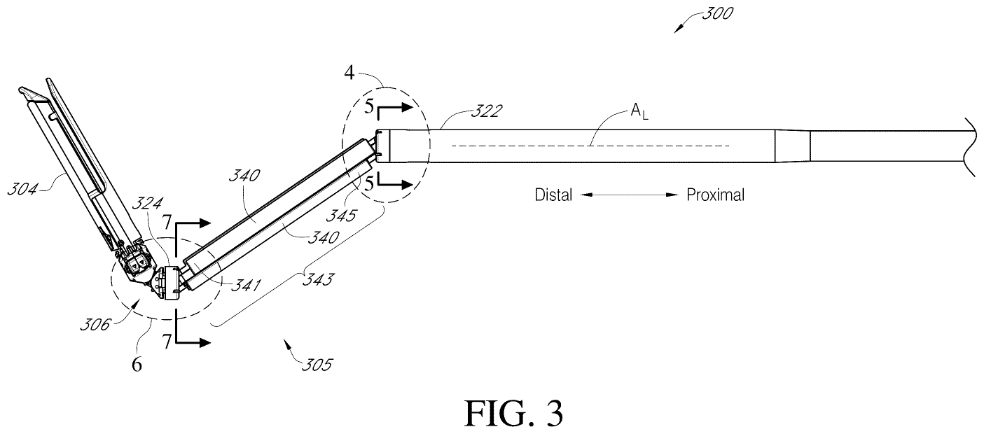

In FIG. 2, the proximal end portion 218 of the first link 214 includes a first transverse link segment 234 extending generally transverse to the longitudinal axis A_{L} when the first link 214 is in a neutral position, and the proximal end portion 226 of the second link 216 includes a second transverse link segment 238 extending generally transverse to the longitudinal axis A_{L} when the second link 216 is in a neutral position. As used herein, the first transverse link segment 234 and second transverse link segment 238 can also be referred to as transverse link segments, and the remaining portions of the first and second link 214, 216 can be referred to as longitudinal link segments. Further, in some exemplary embodiments, such as the embodiment of FIG. 2, the transverse link segments can be formed monolithically with, or otherwise be fixed relative to, the longitudinal link segments. In other embodiments, such as the embodiment of FIGS. 3-7, the transverse link segments can be movable (e.g., rotatable) with respect to the longitudinal link segments.

The first actuation element 230 is coupled to the first transverse link segment 234. The second actuation element 232 is coupled to the second transverse link segment 238. A first moment arm is defined by a distance L₁ from the first pivot axis A₁ to the location the first actuation element 230 is coupled to the first transverse link segment 234 (stated differently the distance is between pivot axis A₁ and the axis of first actuation element 230). Likewise, a second moment arm is defined by a distance L₂ from the second axis A₂ to the location at which the second actuation element 232 is coupled to the second transverse link segment 238 (stated differently the distance is between pivot axis A₂ and the axis of second actuation element 232). In the embodiment of FIG. 2, the first transverse link segment 234 extends from one side of the longitudinal axis A_{L} to an opposite side of the longitudinal axis A_{L}. Likewise, the second transverse link segment 238 extends from one side to the other of the longitudinal axis A_{L}.

The arrangement of the first transverse link segment 234 and second transverse link segment 238 crossing the longitudinal axis A_{L} can be utilized to provide a relatively long lever arm with which each respective actuation element acts on the first and second links 214 and 216. Thus, the force applied to the actuation elements 230 232 (e.g., via a force transmission mechanism such as force transmission mechanism 110 (FIG. 1)) can be multiplied to a greater degree than other arrangements that similarly provide a lateral offset but do not cross the longitudinal axis. In addition, due to the favorable leverage ratio provided by the transverse link segments 234, 238, the articulation mechanism 205 can also exhibit greater resistance to deflection from outside forces or reaction forces generated through actuation of the end effector (e.g., end effector 104 or 204). Furthermore, this arrangement can contribute to overall compact packaging of the articulation mechanism 205, e.g., keeping the overall size of the instrument within a specified range, e.g., 8 mm or less in outer diameter, 10 mm or less outer diameter, 12 mm or less outer diameter, or other sizes and size ranges, while providing favorable multiplication of the forces applied via the actuation elements 230, 232. Other arrangements are considered within the scope of the disclosure, such as the first transverse link segment 234 and second transverse link segment 236 extending from the respective proximal end portions of the first link 214 and second link 216 toward the longitudinal axis A_{L} but not crossing the longitudinal axis A_{L}.

Upon actuation of the first actuation element 230, e.g., application of a tensile (or pulling) force to the first actuation element 230 (while allowing for payout of the second actuation member 232 in a pull-pull type arrangement of actuation members) a moment is generated rotating the first link 214 about the first axis A₁ in direction M₁. Due to the coupling of the first link 214 and second link 216 to the distal portion 224 at respective axes A₃ and A₄, rotation of the first link 214 about the first axis A₁ also causes the second link 216 to rotate about the second axis A₂ in the same direction M₂ if the actuation element 232 is allowed to pay out. Thus, as the first link 214 and second link 216 rotate in a coordinated manner based on tensile force applied to first actuation element 230, the distal portion 224 moves generally laterally in direction D₁ with respect to the longitudinal axis A_{L}, while the proximal portion 222 remains in its generally neutral alignment with respect to the longitudinal axis A_{L}. Stated in a different way, upon articulation of the articulation mechanism 205, the articulation mechanism 205 rotates in a first direction (i.e., M₁) relative to the proximal portion 222, and the distal portion 224 rotates in a direction opposite M₁ relative to the articulation mechanism 205. In other words, a counter pivot of the proximal and distal portions relative to the articulation mechanism occurs.

Similarly, upon application of a tensile (pulling) force to the second actuation element 232 (and a corresponding pay out of the first actuation element 230 in a pull-pull type of arrangement), a moment is generated about the second axis A₂ rotating the second link 216 about the second axis A₂ in a direction opposite Mₛ Due to the coupling of the first link 214 and second link 216 to the distal portion 224 at respective axes A₃ and A₄, the first link 214 rotates about the second axis in the same direction (i.e., opposite M₁), moving the distal portion 224 generally laterally in a direction opposite D₁, thereby providing a lateral offset from side to side as desired.

While application of tensile force to one of actuation element 230, 232 and payout of the other actuation element 230, 232 is discussed herein in the context of a pull-pull arrangement of actuation elements 230, 232, the disclosure is not so limited, and the actuation elements 230, 232 could be replaced with one or more push-pull type actuation elements without modifying the general operation of the disclosed embodiments. Moreover, as further discussed below, it is not necessary to actively pay out or provide a counter force on the other of a pair of actuation elements controlling articulation in a given degree of freedom (e.g., pitch or yaw) and instead force can be actively applied to actuation one actuation element of the pair and the movement of the links in response thereto can cause the counter-pivoting movement and passive actuation of the other actuation element of the pair.

As discussed further herein in connection with other embodiments, the kinematic relationships of the first link 214, the second link 216, the proximal portion 222, and the distal portion 224 may result in movement of the distal portion 224 laterally relative to the longitudinal axis A_{L} but not necessarily confined to a direction precisely perpendicular to the longitudinal axis A_{L}. Accordingly movement of the distal portion 224 may include some degree of rotation of the distal portion 224 relative to the longitudinal axis A_{L} in addition to the lateral movement. In such embodiments, the movement of the distal portion 224 may still be referred to as lateral movement as such lateral movement is a component of the overall movement.

In the arrangement of FIG. 2, each of the first link 214 and second link 216 are rotatable in a single degree of freedom relative to the proximal portion 222, i.e., the first link 214 about the first and third axes A₁ and A₃, and the second link 216 about the second and fourth axes A₂ and A₄. Accordingly, in FIG. 2, the distal portion 224 is moveable within a plane (i.e., the plane of FIG. 2) normal to the axes A₁ and A₂ in a path defined by the kinematic relationships of the first link 214, second link 216, proximal portion 222, and distal portion 224. In other arrangements, additional degrees of freedom of movement of links connecting the proximal portion and the distal portion may be introduced to facilitate movement of the distal portion in three-dimensional space, such as movement in both pitch and yaw. Stated another way, the distal portion can be enabled to move relative to the proximal portion in two or more planes, such as two orthogonal planes.

For example, with reference now to FIGS. 3-7, an instrument 300 including another arrangement of an articulation mechanism (shown actuated to articulate the instrument in various of the figures) that allows a proximal portion and distal portion to articulate relative to each other in multiple degrees of freedom (e.g., pitch and yaw independently or in combination) is illustrated. The articulation mechanism 305 includes four links 343 (two of which are visible in the view of FIG. 3). Like the embodiments of FIGS. 1 and 2, the four links 343 are coupled between a proximal portion 322 of the instrument 300 and distal portion 324 of the instrument 300. Each of the four links 343 comprises a longitudinal link segment 340 having a proximal end portion 345 coupled to the proximal portion 322 and a distal end portion 341 coupled to the distal portion 324 (shown in FIG. 3). As described further below, the four links 343 additionally include transverse link segments 344p positioned at a proximal end portion of the respective links 343, and transverse link segments 344d positioned at a distal end portion of the respective links 343.

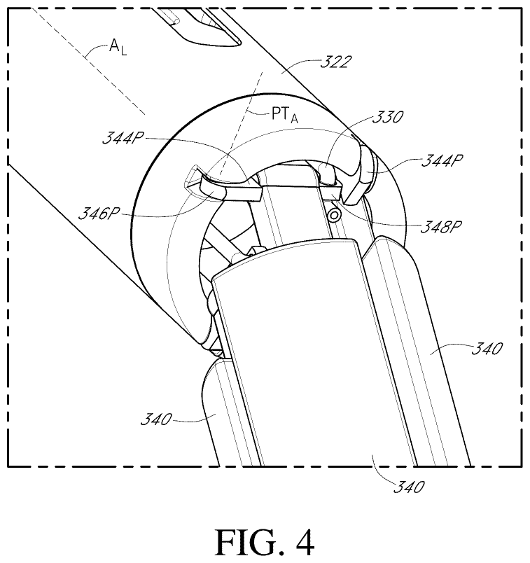

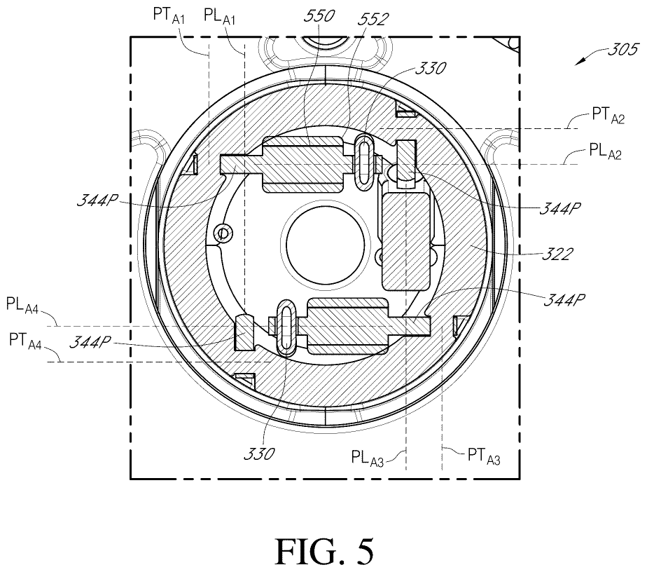

FIG. 4 shows an enlarged view of the proximal portion 322 and the proximal end portion 345 of the longitudinal link segments 340 (three of the links 343 being viewable in the FIG. 4). As can be seen in FIG. 4, each of the four longitudinal link segments 340 is coupled to the proximal portion 322 by a respective transverse link segment 344p that extends generally perpendicular to the longitudinal axis A_{L}. A first end 346p of each transverse link segment 344p is pivotably coupled to the proximal portion 322 (e.g., via a pinned joint or the like) such that the transverse link segment 344p is rotatable relative to the proximal portion 322 about an axis PT_{A} oriented normal to the longitudinal axis A_{L} and offset from the longitudinal axis A_{L} (i.e., the axis PT_{A} and longitudinal axis A_{L} are orthogonal, non-intersecting, and separated (offset) in an orthogonal direction to both axes from each other as further explained below with reference to FIG. 5). A second end 348p of each transverse link segment 344p is coupled to an actuation element 330 and is configured to be actuated by the actuation element 330 to impart motion to the longitudinal link segments 340 as described further below. The actuation elements 330 may be push-pull actuation elements, such as actuation rods that can transfer pushing (e.g., compressive) forces and pulling (e.g., tensile) forces. Alternatively, actuation elements can be of the pull-pull type, such as cables or the like.

FIG. 5 shows a cross-sectional view of the articulation mechanism 305 along section 5-5 of FIG. 3. As illustrated in FIG. 5, the four axes PT_{A1}, PT_{A2}, PT_{A3}, and PT_{A4} about which each of the transverse link segments 344p are rotatable form two sets of two axes opposing one another across the diameter of the instrument 300. Opposing axes PT_{A1} and PT_{A3} are oriented parallel to one another and opposing axes PT_{A2} and PT_{A4} are parallel to another and perpendicular to axes PT_{A1} and PT_{A3}. Each pair of axes PT_{A1} and PT_{A3} and PT_{A2} and PT_{A4} also extend along a direction perpendicular to longitudinal axis A_{L} and are radially offset from the longitudinal axis A_{L}, with each axis of a pair being offset the same distance but on diametrically opposite sides of the longitudinal axis A_{L}. Rotation of opposing pairs of transverse link segments 344p about their respective axes PT_{An} imparts a first degree of freedom of movement (e.g., a respective pitch or yaw articulation) to each of the links 343 (FIG. 4) relative to the proximal portion 322.

The articulation mechanism 305 enables an additional degree of freedom of movement by also rotatably coupling the longitudinal link segments 340 to the transverse link segments 344p. Referring again to FIG. 5, each longitudinal link segment 340 is rotatable relative to its respective transverse link segment 344p along an axis PL_{An} (PLA₁, PLA₂, PLA₃, PLA₄ in FIG. 5) extending longitudinally along each transverse link segment 344p (perpendicular and offset from the longitudinal axis A_{L} in a manner similar to axes PT_{An} described above). In the embodiment of FIGS. 4-7, and most clearly shown in FIG. 5, each transverse link segment 344p includes a bearing surface 550, and each longitudinal link segment 340 includes a bore 552 that receives the bearing surface 550. This allows each transverse link segment 344p coupled to the proximal portion 322 to be rotatably coupled to a proximal end portion 345 of a respective longitudinal link segment 340.

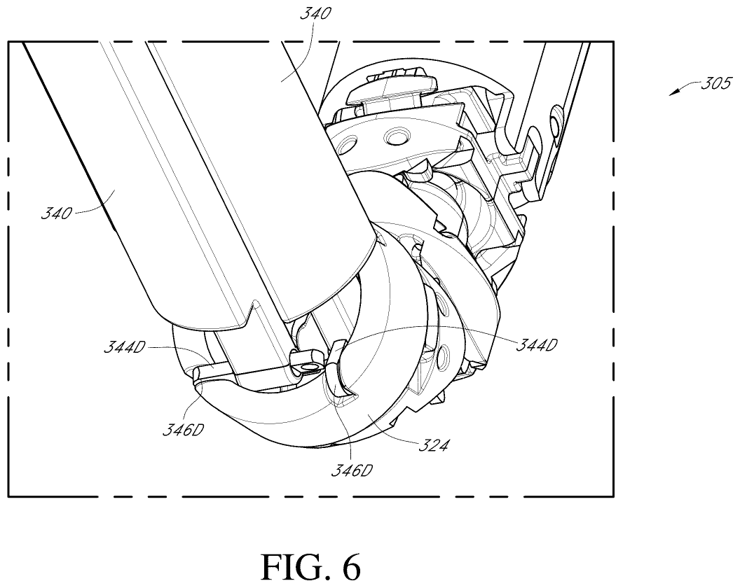

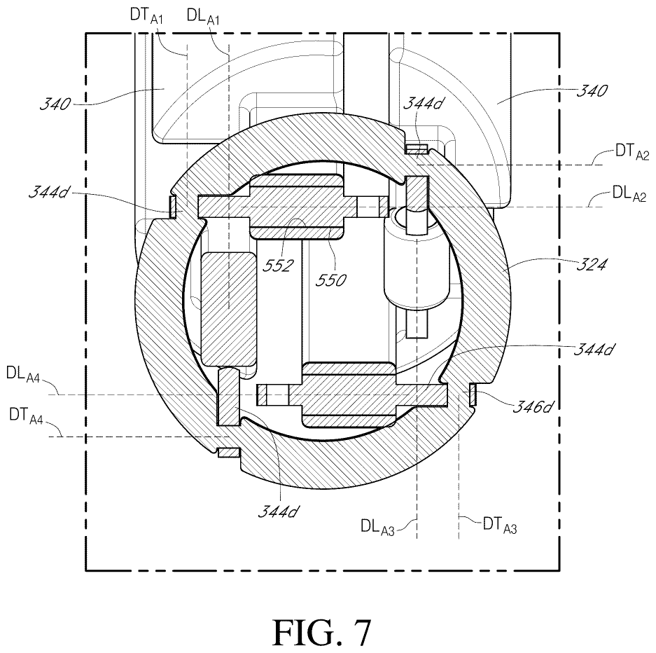

FIG. 6 shows an enlarged view of the distal portion 324 and distal end portions of the links 343 of the articulation mechanism 305. The links 343 include the longitudinal link segments 340 and distal transverse link segments 344d coupled to the distal portion 324 in an arrangement similar to that of the proximal transverse link segments 344p coupled to the proximal portion 322. Each distal transverse link segment 344d includes a first end 346d pivotably coupled (e.g., via a pinned joint or the like) to the distal portion 324 about an axis DT_{An} (FIG. 7). In the embodiment of FIGS. 3-7, each distal transverse link segment 344d coupled at the distal portion 324 is not coupled to any actuation element and is actuated only based on inputs from the actuation elements 330 coupled to the proximal transverse link segments 344p, as discussed further herein.

Referring to FIG. 7, a cross-sectional view along section 7-7 of FIG. 3 is shown. As described above with respect to the proximal transverse link segments, each of the distal transverse link segments 344d includes a bearing surface 550, and each distal end portion 341 of each longitudinal link segment 340 includes a bore 552 that receives a bearing surface 550 of a respective transverse link segment 344d, which allows each longitudinal link segment 340 to be rotatable relative to its respective transverse link segment 344d about a longitudinal axis of the respective transverse link segment. However, as described above, the distal transverse link segments 344d differ from the proximal transverse link segments 344p in that the distal transverse link segments 344d are not coupled to actuation elements. Thereby, the actuation elements 330 are coupled only to the proximal end portions 345 of the longitudinal link segments 340. Because of this arrangement, actuation elements are not required to extend from the proximal end portions 345 of the longitudinal link segments 340 to the distal end portions 341 of the longitudinal link segments 340.

As used herein, an arrangement including a longitudinal link segment 340 and two transverse link segments 344 (i.e., 344p or 344d), one at each of the proximal and distal end portions of the longitudinal link segment 340, can be referred to as a link 343. In the embodiment of FIGS. 3-7, the longitudinal link segment 340 and the pair of transverse link segments 344 are rotatable with respect to one another and comprise separate components, in order to provide additional degrees of freedom as discussed above. However, links of other embodiments of the disclosure, such as links 214 and 216, can include a longitudinal link segment and a transverse link segment that are fixed with respect to one another, and can optionally be formed integrally.

Articulation of the articulation mechanism 305 is accomplished in a manner similar to that discussed in connection with FIG. 2, but with additional possible ranges of movement facilitated by the configuration and coupling (coordinated movement) of transverse link segments 344 (referred to as 344 generally and can include 344p and 344d) and longitudinal link segments 340. For example, of two actuation elements 330 depicted in FIG. 4 coupled to opposing transverse link segments 344 (such as the pair of transverse link segments 344 that rotate about axes PT_{A1} and PT_{A3}, or the pair of transverse link segments 344 that rotate about axes PT_{A2} and PT_{A4}, i.e., an opposed pair of proximal transverse link segments), one actuation element 330 can be pulled or pushed to create the desired articulation motion. It can be appreciated that while only one of the actuation elements 330 needs to be acted on to cause articulation, by coordinated pushing and pulling of two actuation elements 330 that cause opposite articulation in a degree of freedom, a more robust motion can occur. In a manner similar to that discussed in connection with the embodiment of FIG. 2, applying a force (e.g., pushing or pulling) on one of a pair of opposing actuation elements, or coordinated opposing forces (e.g., pushing and pulling) on the pair together as noted above, generates moments about the pair of opposing longitudinal link segments 340 and creates lateral movement of the distal portion 324 relative to the proximal portion 322 with respect to the longitudinal axis AL of the articulation mechanism 305.

For example, upon actuation of the actuation elements 330 coupled with transverse link segments 344p rotatable about axes PT_{A1} and PT_{A3}, the respective proximal transverse link segments 344p rotate about axes PT_{A1} and PT_{A3} at the proximal portion 322. This in turn causes the respective distal transverse link segments 344d to rotate about axes DT_{A1} and DT_{A3} at the distal portion 324. The corresponding rotations of the transverse link segments 344p and 344d cause lateral translational movement of the distal portion 324 relative to the longitudinal axis A_{L} (FIG. 3). At the same time, longitudinal link segments 340 coupled at the proximal end portions 345 thereof to proximal transverse link segments 344 (which pivot about axes PT_{A2} and PT_{A4} at the proximal portion 322) and coupled at the distal end portions 341 thereof to transverse link segments 344 (which pivot about axes DT_{A2} and DT_{A4} at the distal portion 324) rotate about each of the associated bearing surfaces 550. Because each of the longitudinal link segments 340 is pivotably coupled to its associated transverse link segments 344 at the proximal and distal end portions 341, 345 of each of the longitudinal link segment 340, as one opposing pair of links 343 are actuated via actuation elements 330, the longitudinal link segments 340 of the other opposing pair passively rotate about their associated transverse link segments 344 on bearing surfaces 550.

In this way, the articulation mechanism 305 can generate movement in two orthogonal planes based on inputs from the four actuation elements 330. In some embodiments, independent movements in each of the two orthogonal planes may be assigned a kinematic label, e.g., movement in a first plane is pitch motion while movement in a second plane is yaw motion. Further, because of the arrangement of the coupling and coordinated movement caused by actuation of the links 343 at the proximal portion 322, the passive movement caused at distal portion 324 is constrained, which allows the coordinated articulation movement to be achieved without the need for actuation elements to extend through the articulation mechanism, which can in turn allow for more rigid actuation elements to be used if desired (e.g., push-pull rods or the like) with greater ability to transmit compressive loads.

The arrangement of the articulation mechanisms according to exemplary embodiments of the disclosure enables the actuation elements 330 to terminate distally at the proximal portion of the linkage system. Compared to other articulation mechanism designs, such as joints in which actuation elements extend through the joint from the proximal end to the distal end, the present design can contribute to robustness of the articulation mechanism by preventing contact between environmental structures or materials and the actuation elements. Further, by coupling the actuation elements 330 at the proximal end such that the actuation elements have a greater mechanical advantage to move the link components compared to other articulation mechanism designs, the articulation mechanisms are better able to resist push and pull forces applied to the end effector without deflection that can occur in some articulation mechanism configurations.

In some embodiments, such as those discussed above in connection with FIGS. 2-7, the respective pairs of axes about which the longitudinal link segments rotate with respect to the proximal portions and distal portions are positioned equidistant from, but on opposite sides of, the longitudinal axis A_{L} in the neutral unarticulated state of the proximal and distal portions relative to each other With this arrangement, as the articulation mechanism is articulated, the longitudinal axis of the distal portion can maintain its same orientation with respect to the longitudinal axis of the proximal portion prior to articulation. In other words as the longitudinal axis of the distal portion was oriented parallel to the longitudinal axis of the proximal portion, that relative orientation can be retained throughout the articulation.

In other embodiments, the kinematic relationships between the proximal portion, distal portion, and links can be altered to introduce a desired amount of rotation of the distal portion relative to the proximal portion (i.e., reorientation of the longitudinal axis of the distal portion relative to the proximal portion) throughout the range of motion of the articulation mechanism. For example, as noted above, the articulation mechanism may be used to alter the path of the instrument shaft distal portion from a longitudinal axis to change an angle at which the end effector approaches the remote site (such as a surgical site) from an access cannula.

Referring now to FIG. 8, another articulation mechanism 805 according to an embodiment of the present disclosure is shown. For simplicity in illustration and discussion, the articulation mechanism 805 is shown as having a range of motion in a single plane similar to the embodiment of FIG. 2. However, the aspects discussed in connection with the embodiment of FIG. 8 also have applicability to the embodiments of FIGS. 3-7 and other embodiments of the present disclosure, e.g., embodiments in which the articulation mechanism is independently moveable in two orthogonal planes. The articulation mechanism 805 includes a proximal portion 822, a distal portion 824, and first and second links 814 and 816. The first link 814 is rotatably coupled to the proximal portion 822 at a proximal end portion 818 of the first link 814 at a first axis A₁. A distal end portion 820 of the first link 814 is coupled to the distal portion 824 at a third axis A₃. The second link 816 is rotatably coupled to the proximal portion 822 at a proximal end portion 826 of the second link 816 at a second axis A₂. A distal end portion 828 of the second link 816 is coupled to the distal portion 824 about a fourth axis A₄. Other components of the articulation mechanism 805, including actuation elements 830, 832, and transverse link segments 834, 838 are arranged similarly to the embodiment of FIG. 2.

As can be seen in FIG. 8, axes A₁ and A₂ are each offset from the longitudinal axis A_{L} by a distance D₁, are parallel to each other, and extend in a direction perpendicular to the longitudinal axis A_{L} Axes A₃ and A₄ also are reach offset from the longitudinal axis A_{Ld} (i.e., a longitudinal axis of the distal portion 824 through which the longitudinal axis A_{L} of the linkage system passes when the articulation mechanism 805 is in a neutral, unarticulated state) by a second distance D₂ different from the first distance D₁. Axes A₃ and A₄ also extend parallel to each other and perpendicular to axis A_{Ld}. In the embodiment of FIG. 8, the second distance D₂ is less than the distance D₁. Due to the kinematic relationships created by the second distance D₂ being shorter than the first distance D₁, when the articulation mechanism 805 is articulated as shown in FIG. 8 to move the distal portion 824 laterally away from the longitudinal axis A_{L}, the distal portion 824 rotates about A₃ and A₄ in direction R back toward the longitudinal axis A_{L}. This arrangement can reduce the overall range of motion required for other articulation mechanism (such as wrist mechanisms, e.g., a wrist mechanism similar to wrist mechanism 106 discussed in connection with FIG. 1)) distal of the articulation mechanism 805 by automatically directing the end effector (e.g., end effector 104 in FIG. 1) back toward the longitudinal axis A_{L}.

In various embodiments, the D1 distances can be equal or unequal, and likewise the D2 distances, but regardless, the relative difference is what produces the desired outcome (i.e., (D1+D1)≠(D2+D2)).

Articulation mechanisms according to embodiments disclosed herein provide movement in one or both of pitch and yaw while providing robustness, resistance to deflection due to externally applied forces, and reduced likelihood of actuation elements experiencing interference with environmental structures or materials. Additionally, articulation mechanisms according to embodiments disclosed herein can have kinematic characteristics that reduce a required range of motion of other articulation mechanisms of an instrument, such as a wrist mechanism.

Embodiments described herein may be used, for example, with remotely operated, computer-assisted systems, such, for example, teleoperated surgical systems. Further, embodiments described herein may be used, for example, with a da Vinci® Surgical System, such as the da Vinci Si® Surgical System, da Vinci X® Surgical System, the da Vinci Xi® Surgical System, all with or without Single-Site® single orifice surgery technology, or the da Vinci SP® Surgical System, all commercialized by Intuitive Surgical, Inc., of Sunnyvale, California.

The embodiments described herein are not limited to the surgical systems noted above, and various other teleoperated, computer-assisted surgical system configurations may be used with the embodiments described herein. Further, although various embodiments described herein are discussed in connection with a manipulating system of a teleoperated surgical system, the present disclosure is not limited to use with a teleoperated surgical system. Various embodiments described herein can optionally be used in conjunction with hand-held, manual instruments.

As discussed above, in accordance with various embodiments, instruments of the present disclosure are configured for use in teleoperated, computer-assisted surgical systems employing robotic technology (sometimes referred to as robotic surgical systems or teleoperated surgical systems). Referring now to FIG. 9, an embodiment of a manipulator system 1900 of a computer-assisted surgical system, to which medical instruments are configured to be mounted for use, is shown. Such a surgical system may further include a user control system, such as a surgeon console (not shown) for receiving input from a user to control instruments coupled to the manipulator system 1900, as well as an auxiliary system, such as auxiliary systems associated with the da Vinci® systems noted above.

As shown in the embodiment of FIG. 9, a manipulator system 1900 includes a base 1920, a main column 1940, and a main boom 1960 connected to main column 1940. Manipulator system 1900 also includes a plurality of manipulator arms 1910, 1911, 1912, 1913, which are each connected to main boom 1960. Manipulator arms 1910, 1911, 1912, 1913 each include an instrument mount portion 1922 to which a medical instrument 1930 may be mounted, which is illustrated as being attached to manipulator arm 1910. While the manipulator system 1900 of FIG. 9 is shown and described having a main boom 1960 to which the plurality of manipulator arms are coupled and supported thereby, in other embodiments, the plurality of manipulator arms can be coupled and supported by other structures, such as an operating table, a ceiling, wall, or floor of an operating room, etc.

Instrument mount portion 1922 comprises a drive assembly 1923 and a cannula mount 1924, with a transmission mechanism 1934 (which may generally correspond to the transmission mechanism 110 discussed in connection with FIG. 1) of the instrument 1930 connecting with the drive assembly 1923, according to an embodiment. Cannula mount 1924 is configured to hold a cannula 1936 through which a shaft 1932 of instrument 1930 may extend to a surgery site during a surgical procedure. Drive assembly 1923 contains a variety of drive and other mechanisms that are controlled to respond to input commands at the surgeon console and transmit forces to the transmission mechanism 1934 to actuate the instrument 1930. Although the embodiment of FIG. 9 shows an instrument 1930 attached to only manipulator arm 1910 for ease of viewing, an instrument may be attached to any and each of manipulator arms 1910, 1911, 1912, 1913.

Other configurations of surgical systems, such as surgical systems configured for single-port surgery, are also contemplated. For example, with reference now to FIG. 10, a portion of an embodiment of a manipulator arm 2140 of a manipulator system with two medical instruments 2300, 2310 in an installed position is shown. The instruments 2300, 2310 can generally correspond to instruments discussed above, such as instrument 100 disclosed in connection with FIG. 1. For example, the embodiments described herein may be used with a da Vinci SP® Surgical System, commercialized by Intuitive Surgical, Inc. of Sunnyvale, California. The schematic illustration of FIG. 10 depicts only two medical instruments for simplicity, but more than two medical instruments may be mounted in an installed position at a manipulator system as those having ordinary skill in the art are familiar with. Each instrument 2300, 2310 includes a shaft 2320, 2330 that at a distal end has a moveable end effector or an endoscope, camera, or other sensing device, and may or may not include a wrist mechanism (not shown) to control the movement of the distal end.

In the embodiment of FIG. 10, the distal end portions of the instruments 2300, 2310 are received through a single port structure 2380 to be introduced into the patient. As shown, the port structure includes a cannula and an instrument entry guide inserted into the cannula. Individual instruments are inserted into the entry guide to reach a surgical site.

Other configurations of manipulator systems that can be used in conjunction with the present disclosure can use several individual manipulator arms. In addition, individual manipulator arms may include a single instrument or a plurality of instruments. Further, as discussed above, a medical instrument may be an instrument with an end effector or may be a camera instrument or other sensing instrument utilized during a medical procedure to provide information, (e.g., visualization, electrophysiological activity, pressure, fluid flow, and/or other sensed data) of a remote surgical site.

Transmission mechanisms 2385, 2390 (which may generally correspond to transmission mechanism 110 disclosed in connection with FIG. 1) are disposed at a proximal end of each shaft 2320, 2330 and connect optionally through a sterile adaptor 2400, 2410 with drive assemblies 2420, 2430. Drive assemblies 2420, 2430 contain a variety of internal mechanisms (not shown) that are controlled by a controller (e.g., at a control cart of a teleoperated surgical system) to respond to input commands at a surgeon side console of a surgical system to transmit forces to the transmission mechanisms 2385, 2390 to actuate instruments 2300, 2310.

The embodiments described herein are not limited to the embodiments of FIG. 9 and FIG. 10, and various other teleoperated, computer-assisted surgical system configurations may be used with the embodiments described herein. The diameter or diameters of an instrument shaft and end effector are generally selected according to the size of the cannula with which the instrument will be used and depending on the medical procedures being performed.

This description and the accompanying drawings that illustrate various embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures may represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to another embodiment, the element may nevertheless be claimed as included in the other embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

What is claimed is:

1. A medical instrument comprising:
a proximal portion, a distal portion, and an articulation mechanism coupling the proximal portion to the distal portion,
a longitudinal axis extending along the proximal portion;
wherein the articulation mechanism comprises:
a first link comprising a proximal end portion pivotably coupled to the proximal portion at a first pivot axis and a distal end portion pivotably coupled to the distal portion;
a first actuation element coupled to the proximal end portion of the first link and extending proximal to the first link along the longitudinal axis and configured to transmit actuation forces to the proximal end portion of the first link;
a second link comprising a proximal end portion pivotably coupled to the proximal portion at a second pivot axis and a distal end portion pivotably coupled to the distal portion; and
a second actuation element coupled to the distal end portion of the second link and extending proximal to the second link along the longitudinal axis and configured to transmit actuation forces to the proximal end portion of the second link;
wherein the first link is pivotable relative to the proximal portion about the first pivot axis,
wherein the first actuation element is offset from the first pivot axis such that actuation of the first actuation element creates a moment to rotate the first link about the first pivot axis and causes rotation of the first link about the proximal portion,
wherein the second link is pivotable relative to the proximal portion about the second pivot axis, and
wherein the second actuation element is offset from the second pivot axis such that actuation of the second actuation element creates a moment to rotate the second link about the second pivot axis and causes rotation of the second link about the proximal portion.

2. The medical instrument of claim 1,
wherein the first link is pivotably coupled to the distal portion about a third pivot axis and the second link is pivotably coupled to the distal portion about a fourth pivot axis, and
wherein the first pivot axis and the second pivot axis are offset from the longitudinal axis by a first distance and the third pivot axis and the fourth pivot axis are offset from the longitudinal axis by a second distance.

3. The medical instrument of claim 2, wherein the first distance and the second distance are equal.

4. The medical instrument of claim 2, wherein the first distance is greater than the second distance.

5. The medical instrument of claim 1,
wherein the first link comprises a first transverse link segment extending generally perpendicular to the longitudinal axis in a neutral, unarticulated state of the articulation mechanism, and
wherein the first actuation element is coupled to the first transverse link segment.

6. The medical instrument of claim 5, wherein the first transverse link segment extends from a proximal end portion of the first link.

7. The medical instrument of claim 5, wherein the first transverse link segment extends away from the first pivot axis and toward the longitudinal axis.

8. The medical instrument of claim 5,
wherein the second link comprises a second transverse link segment extending generally perpendicular to the longitudinal axis, and
wherein the second actuation element is coupled to the second transverse link segment.

9. The medical instrument of claim 8, wherein the second transverse link segment extends from a proximal end portion of the second link.

10. The medical instrument of claim 8, wherein the second transverse link segment extends from the second pivot axis toward the longitudinal axis.

11. The medical instrument of claim 1,
wherein the proximal end portion of each of the first link and the second link comprises a proximal transverse link segment,
wherein the distal end portion of each of the first link and the second link comprises a distal transverse link segment,
wherein each of the first link and the second link comprises
a longitudinal link segment rotatably coupled to each of the proximal transverse link segment and the distal transverse link segment.

12. The medical instrument of claim 11, wherein the proximal transverse link segment is rotatably coupled to the longitudinal link segment along a proximal transverse link pivot axis orthogonal to the first pivot axis.

13. The medical instrument of claim 1, further comprising:
a third link comprising a proximal end portion pivotably coupled to the proximal portion at a third pivot axis and a distal end portion pivotably coupled to the distal portion;
a third actuation element coupled to the proximal end portion of the third link and extending proximal to the third link along the longitudinal axis and configured to transmit actuation forces to the proximal end portion of the third link;
a fourth link comprising a proximal end portion pivotably coupled to the proximal portion at a fourth pivot axis and a distal end portion pivotably coupled to the distal portion; and
a fourth actuation element coupled to the distal end portion of the fourth link and extending proximal to the fourth link along the longitudinal axis and configured to transmit actuation forces to the proximal end portion of the fourth link;
wherein the third link is pivotable relative to the proximal portion about the third pivot axis,
wherein the third actuation element is offset from the third pivot axis such that actuation of the third actuation element creates a moment to rotate the third link about the third pivot axis and causes rotation of the third link about the proximal portion,
wherein the fourth link is pivotable relative to the proximal portion about the fourth pivot axis, and
wherein the fourth actuation element is offset from the fourth pivot axis such that actuation of the fourth actuation element creates a moment to rotate the fourth link about the fourth pivot axis and causes rotation of the fourth link about the proximal portion.

14. The medical instrument of claim 13,
wherein the first and second pivot axes are parallel to each other and perpendicular to the longitudinal axis,
wherein the third and fourth pivot axes are parallel to each other and perpendicular to the longitudinal axis, and to the first and second pivot axes.

15. The medical instrument of claim 14, wherein the distal portion is movable relative to the proximal portion in pitch and yaw.

16. The medical instrument of claim 1,
wherein the rotation of the first link and second link about the first pivot axis and the second pivot axis, respectively, causes translation of the distal portion.

17. A medical instrument, comprising:
a shaft comprising:
a proximal portion defining a longitudinal axis;
a distal portion; and
an articulation mechanism coupling the proximal portion and the distal portion, wherein the articulation mechanism comprises a plurality of links rotatably coupled at respective proximal pivot axes to the proximal portion and at respective distal pivot axes to the distal portion, the proximal and distal pivot axes being perpendicular to and radially offset from the longitudinal axis; and
a plurality of actuation elements coupled to each of the plurality of links, respectively, the plurality of actuation elements terminating at and extending proximally from a proximal portion of the plurality of links;
wherein actuation of an actuation element of the plurality of actuation elements causes pivoting of the articulation mechanism in a given direction relative to the proximal portion and pivoting of the distal portion relative to the articulation mechanism in a direction opposite the given direction.

18. The medical instrument of claim 17, wherein the articulation mechanism is configured to move the distal portion in translation relative to the proximal portion.

19. The medical instrument of claim 17, wherein the articulation mechanism is configured to move the distal portion in rotation relative to the proximal portion.

20. An articulation mechanism for coupling a proximal portion of an elongate instrument to a distal portion of an elongate instrument, the articulation mechanism comprising:
a first link comprising:
a proximal end portion pivotably coupled to the proximal portion at a first location radially offset in a first direction from a longitudinal axis of the proximal portion, and
a distal end portion pivotably coupled to the distal portion at a second location radially offset in the first direction from the longitudinal axis;
a first actuation element coupled to the proximal end portion of the first link and configured to transmit force to create a moment to rotate the first link about a first axis of rotation at the first location and thereby cause rotation of the first link about a second axis of rotation at the second location;
a second link comprising:
a proximal end portion pivotably coupled to the proximal portion at a third location radially offset in a second direction, opposite the first direction, from the longitudinal axis, and
a distal end portion pivotably coupled to the distal portion at a fourth location radially offset in the second direction from the longitudinal axis; and
a second actuation element coupled to the proximal end portion of the second link and configured to transmit force to create a moment to rotate the second link about a third axis of rotation extending through the third location and thereby cause rotation about a fourth axis of rotation extending through the fourth location;
wherein the first, second, third, and fourth axes of rotation extend parallel to each other and perpendicular to the longitudinal axis.
